(19) United States
(12) Patent Application Publication
(10) Pub. No.: US 2026/0349237 A1
(43) Pub. Date: Nov. 5, 2026

(54) 3D VIDEO ENDOSCOPE

(71) Applicant: Blazejewski MEDI-TECH GmbH, Sexau (DE)

(72) Inventor: Reinhold BLAZEJEWSKI, Gutach i. Br. (DE)

(73) Assignee: Blazejewski MEDI-TECH GmbH, Sexau (DE)

(21) Appl. No.: 19/488,926

(22) PCT Filed: Jun. 4, 2024
(86) PCT No.: PCT/DE2024/100504

§ 371 Date: Dec. 1, 2025

(30) Foreign Application Priority Data
Jun. 13, 2023 (DE) 10 2023 115 389.7

(51) Int. Cl. G02B 23/24; A61B 1/00; H04N 13/239; H04N 23/50; H04N 23/55

(52) U.S. Cl. CPC G02B 23/2415; A61B 1/00096; A61B 1/00193; H04N 13/239; H04N 23/55; H04N 23/555; H04N 2213/001

(57) ABSTRACT

A 3D video endoscope has an endoscope shaft designed as a flexible or rigid, elongated hollow body, which has a distal end section with a geometric end section longitudinal axis. The endoscope includes a left optical channel, which has a left objective, a left optical image-transmission system with a left deflection prism, and a left image sensor, and a right optical channel including a right objective, a right optical image-transmission system with a right deflection prism, and a right image sensor. Each deflection prism includes two deflection surfaces at which a light beam incident parallel to the geometric end section longitudinal axis is deflected by 90°, such that the light beam runs perpendicular to the geometric end section longitudinal axis. The deflection prisms are joined together at their respective second deflection surfaces to form a deflection prism unit arranged between the left image sensor and the right image sensor.

The invention relates to a 3D video endoscope with an endoscope shaft designed as a flexible or rigid, elongated hollow body and with a left optical channel and a right optical channel, each of which comprises an objective, an image transmission system, and an image sensor, wherein the objectives, image transmission systems, and image sensors of the left and right optical channels are arranged in a distal end section of the endoscope shaft.

Video endoscopes are used in both technical and medical fields. They are used to examine structures on the surface or in hard-to-reach cavities, channels, or recesses. These structures are often not visible to the naked eye. In the medical field, video endoscopes are used in minimally invasive surgery for examination purposes or in combination with surgical instruments for operations under visual control. An illumination system can be used to illuminate the structure to be examined. The light generated by an external light source is usually guided to the structure to be examined via optical fibers. An imaging system is used to capture the information contained in the light reflected by the structure as an image. An image converter chip, such as CMOS or CCD, is often used as a camera or image sensor. The image sensor, also known as an image generator, converts the optical signals into electrical signals, which are then processed and made visible on a screen or monitor.

Various methods and devices are known for providing the user with as clear as possible impression of the location of the distal end of the endoscope. 3D video endoscopes have a left and a right optical channel. The left channel captures left images and the right channel captures right images. These left and right images are combined into three-dimensional images using an image processing device These three-dimensional images generated by the 3D video endoscope are displayed to a user on a viewing device, such as a monitor or screen. The images are displayed on the viewing device in such a way that the viewer obtains a three-dimensional impression of the location. For this purpose, a screen displays separate images for the viewer's right and left eyes. The viewer usually needs special glasses so that the images intended for the viewer's left eye are only perceived by the left eye, and the images intended for the viewer's right eye are only perceived by the right eye. These include, for example, polarizing filter glasses, color filter glasses, interference filter glasses, and LCD shutter glasses. In addition, there are special viewing devices that the viewer places on their head in close proximity to their eyes. Such viewing devices are integrated into a 3D headset, for example. They are also known as 3D video glasses and are equipped with two displays.

The endoscope comprises a flexible or rigid shaft that is designed as an elongated hollow body. The image sensors are preferably located in a distal end section of the endoscope shaft. The end section of the endoscope shaft has a distal end, a proximal end, and a geometric end section longitudinal axis extending from the distal end to the proximal end. The light reflected by a structure to be examined is coupled in at the distal end via an objective and brought to the image sensor via an image transmission system with optical components such as lenses and prisms or via fiber optics. 3D video endoscopes have a left and a right optical channel, each of which comprises an objective, an image transmission system, and an image sensor: the left optical channel comprises a left objective, a left image transmission system, and a left image sensor, while the right optical channel comprises a right objective, a right image transmission system, and a right image sensor. The left optical image transmission system transmits the image coupled in via the left objective to the left image sensor. The right optical image transmission system transmits the image coupled in via the right objective to the right image sensor. The left and right objectives are usually the same size and are arranged next to each other at the distal end of the distal end section of the endoscope shaft. The left and right optical channels are usually identical. The images captured by the left image sensor and the right image sensor are combined into a three-dimensional image by an image processing device and displayed on a viewing device for the user to see.

If the 3D endoscope is to be used to examine structures in small cavities, the diameter of the shaft must be as small as possible, in particular smaller than the cavity into which the endoscope is to be inserted. With a small shaft diameter, the distance between the left objective and the right objective is small. Care must be taken to ensure that, despite the small distance between the objectives, the overlap area of the fields of view of the two objectives is as large as possible, since a three-dimensional representation of the structures is only possible in the overlap area. In addition, a small shaft diameter means that the diameter of the left and right objectives must be small, in particular smaller than the radius of the shaft. The left image sensor and the right image sensor must also be arranged in the distal end section of the endoscope shaft. In order to obtain the best possible resolution and to be able to image as large an area as possible, the image sensors must be of a certain size. However, due to the small diameter, there is relatively little space available in the end section of the endoscope shaft.

The object of the invention is to provide a 3D video endoscope in which the left and right image transmission systems and the left and right image sensors are arranged in the distal end section of the endoscope shaft in such a way that three-dimensional images with high resolution and a large image area can be generated even with small shaft diameters.

This object is achieved by a 3D video endoscope with the features of claim 1. It is characterized in that the left optical channel has a left deflection prism and the right optical channel has a right deflection prism, each of the two deflection prisms comprising two deflection surfaces. At a first deflection surface, a light beam incident parallel to the geometric longitudinal axis of the end section is deflected by 90°. The light beam deflected in this way is deflected again by 90° at a second deflection surface, so that the light beam runs perpendicular to the geometric longitudinal axis of the end section after deflection at the second deflection surface. The left deflection prism and the right deflection prism are joined together at the left second deflection surface and at the right second deflection surface to form a deflection prism unit. The left image sensor and the right image sensor are arranged parallel to the geometric end section longitudinal axis in such a way that the deflection prism unit is located between the left and right image sensors. Since the left image sensor and the right image sensor are not aligned perpendicular to the end section longitudinal axis but parallel to it, the image sensors can have a large extension in the direction of an end section longitudinal axis without this having any influence on the diameter of the end section of the endoscope shaft. This arrangement is made possible by a double beam deflection through the two deflection prisms in each of the two optical channels. The left and right deflection prisms and the left and right image sensors are assembled in a sandwich-like manner, such that the two deflection prisms are joined together along their respective second deflection surfaces to form a deflection prism unit, and this deflection prism unit is arranged between the two image sensors. This enables a space-saving, compact design. The assembly formed by the left deflection prism, right deflection prism, left image sensor, and right image sensor has a smaller extension in a plane perpendicular to the longitudinal axis of the end section than an arrangement in which the two image sensors are located between the left deflection prism and the right deflection prism. In addition, the electrical connections of the left and right image sensors are arranged on the side of the image sensors facing away from the deflection prisms, so that they are accessible from the outside even when the image sensors and deflection prisms are assembled.

The left deflection prism and the right deflection prism are assembled so that the left second deflection surface lie against the right second deflection surface. Preferably, the left second deflection surface and the right second deflection surface have the same shape and size. They lie completely against each other so that they are aligned.

According to an advantageous embodiment of the invention, the left image sensor with its light-sensitive sensor surface is arranged on the surface of the deflection prism unit in such a way that the light-sensitive sensor surface touches the surface of the deflection prism unit. In addition, the right image sensor with its light-sensitive sensor surface is arranged in such a way that the light-sensitive sensor surface touches the surface of the deflection prism unit. The surface of the deflection prism unit that the left image sensor touches is the exit surface of the left deflection prism. This is referred to as the left exit surface. The light from the left deflection prism is coupled out at this left exit surface. Since the left image sensor with its light-sensitive sensor surface touches the left exit surface, the light passes directly from the left deflection prism to the left image sensor. The same applies to the right deflection prism and the right image sensor: the surface of the deflection prism unit that touches the right image sensor is the exit surface of the right deflection prism. This is referred to as the right exit surface. The light from the right deflection prism is coupled out at this right exit surface. Since the right image sensor with its light-sensitive sensor surface touches the right exit surface, the light passes directly from the right deflection prism into the right image sensor. This minimizes losses and supports a compact design.

According to a further advantageous embodiment of the invention, the left image sensor and the right image sensor are glued to the deflection prism unit.

According to a further advantageous embodiment of the invention, the deflection prism unit, the left image sensor, and the right image sensor are combined to form a deflection prism image sensor unit. This deflection prism image sensor unit can be assembled before installation in the endoscope shaft. This facilitates assembly.

According to a further advantageous embodiment of the invention, the left entrance surface of the left deflection prism faces the left objective lens. Furthermore, the left exit surface of the left deflection prism faces the left image sensor.

According to a further advantageous embodiment of the invention, the left entrance surface of the left deflection prism has the shape of a trapezium comprising two right angles α_{L}=β_{L}=90°, an angle γ_{L}=45° and an angle δ_{L}=135°. The side of the trapezoid that forms one leg of the angles γ_{(L)} and δ_{(L)} delimits the left second deflection surface.

According to a further advantageous embodiment of the invention, the right entrance surface of the right deflection prism faces the right objective lens. Furthermore, the right exit surface of the right deflection prism faces the right image sensor.

According to another advantageous embodiment of the invention, the right entrance surface of the right deflection prism has the shape of a trapezoid, which comprises two right angles α_{R}=β_{R}=90°, an angle γ_{R}=45° and an angle δ_{R}=135°. The side of the trapezoid that forms one leg of the angles γ_{R} and δ_{R} delimits the right second deflection surface.

According to a further advantageous embodiment of the invention, the left entrance surface of the left deflection prism and the right entrance surface of the right deflection prism lie in a common geometric entrance surface plane. The two entrance surfaces together form a rectangle.

According to a further advantageous embodiment of the invention, the left entrance surface of the left deflection prism and the left first deflection surface form an angle ϵ_{L}=45°.

According to a further advantageous embodiment of the invention, the left entrance surface of the left deflection prism and the left second deflection surface form an angle η_{L}=90°.

According to a further advantageous embodiment of the invention, the left entrance surface of the left deflection prism and the left exit surface of the left deflection prism are perpendicular to each other.

According to a further advantageous embodiment of the invention, the right entrance surface of the right deflection prism and the right first deflection surface form an angle ϵ_{R}=45°.

According to a further advantageous embodiment of the invention, the right entrance surface of the right deflection prism and the right second deflection surface form an angle η_{R}=90°.

According to a further advantageous embodiment of the invention, the right entrance surface of the right deflection prism and the right exit surface of the right deflection prism are perpendicular to each other.

According to a further advantageous embodiment of the invention, the left entrance surface of the left deflection prism and the right exit surface of the right deflection prism are parallel to each other in the deflection prism unit.

According to a further advantageous embodiment of the invention, the left first deflection surface and the right first deflection surface in the deflection prism unit are perpendicular to each other.

According to a further advantageous embodiment of the invention, the left entrance surface of the left deflection prism is perpendicular to the geometric longitudinal axis of the end section of the 3D video endoscope. Furthermore, the right entrance surface of the right deflection prism is perpendicular to the geometric longitudinal axis of the end section of the 3D video endoscope.

According to another advantageous embodiment of the invention, the deflection prism unit has the external shape of a mathematical cylinder. This shape supports a compact design.

According to a further advantageous embodiment of the invention, the left deflection prism and the right deflection prism are identical in shape and size.

According to a further advantageous embodiment of the invention, the left deflection prism ensures total reflection of the light coupled at the entrance surface at the left first deflection surface and at the left second deflection surface. This minimizes losses in the left optical channel.

According to a further advantageous embodiment of the invention, the right deflection prism ensures total reflection at the right first deflection surface and at the right second deflection surface. This minimizes losses in the right optical channel.

According to a further advantageous embodiment of the invention, the left deflection prism and the right deflection prism are glued together. This enables an exact, cost-effective, and easy connection of the two deflection prisms.

According to a further advantageous embodiment of the invention, the 3D video endoscope is provided with an image processing device which generates three-dimensional images from left images produced by the left image sensor and right images produced by the right image sensor.

Further advantages and advantageous embodiments of the invention can be found in the following description, the drawing, and the claims.

### DRAWING

The drawing shows an example of the invention. It shows:

FIG. 1 3D video endoscope in longitudinal section,

FIG. 2 Deflection prism image sensor unit of the 3D video endoscope according to FIG. 1 in perspective view,

FIG. 3 Deflection prism image sensor unit according to FIG. 2 in a top view of the entrance surfaces of the deflection prisms, which corresponds to a view from below,

FIG. 4 Deflection prism unit of the deflection image sensor unit according to FIG. 2 in a top view of the entrance surfaces of the left deflection prism and the right deflection prism, which corresponds to a view from below,

FIG. 5 Deflection prism image sensor unit according to FIG. 2 in a side view,

FIG. 6 Top view of the distal end section of the 3D video endoscope according to FIG. 1,

FIG. 7 FIG. 2 in exploded view,

FIG. 8 Deflection prism image sensor unit according to FIG. 2 in a view from above, with and without beam path,

FIG. 9 Deflection prism unit according to FIG. 4 in a side view, with and without beam path.

### DESCRIPTION OF THE EMBODIMENT

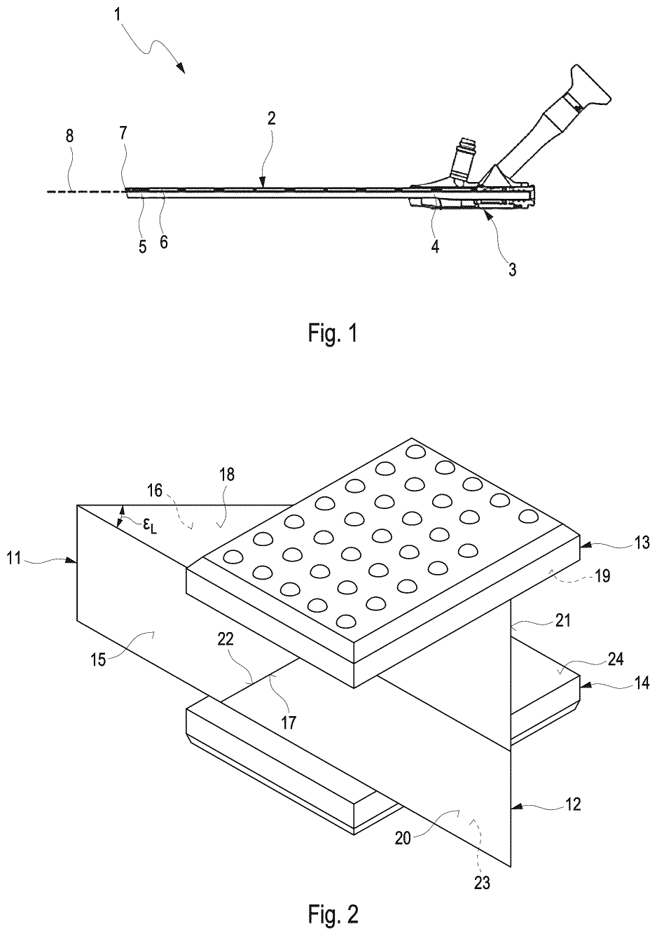

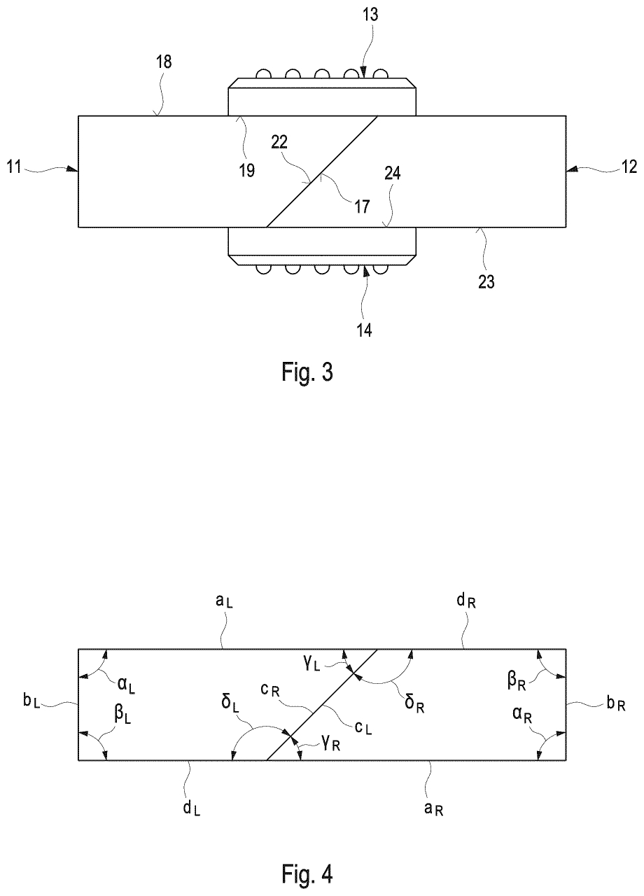

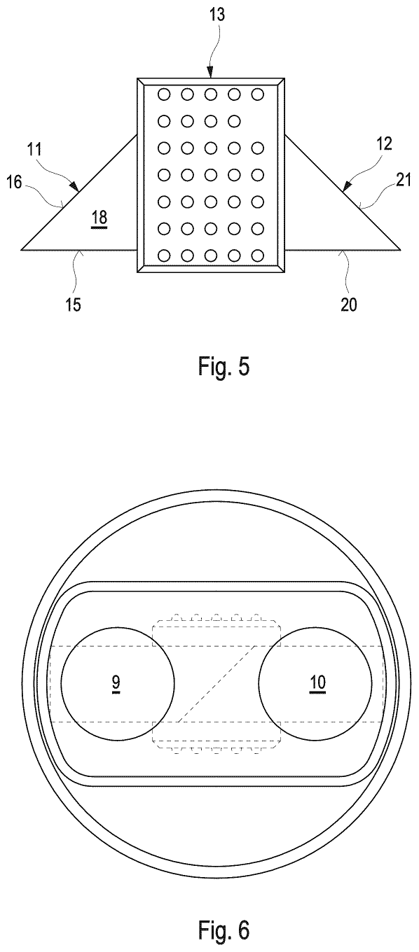

FIGS. 1 to 9 show an example of a 3D video endoscope 1. The 3D video endoscope 1 shown in FIG. 1 in side view comprises an elongated endoscope shaft 2 and an endoscope base body 3. The endoscope shaft 2 is received with its proximal shaft end 4 in the endoscope base body 3. At its other end, the endoscope shaft 2 has a distal end section 5. The distal end section 5 comprises a proximal end section end 6 and a distal end section end 7. A geometric end section longitudinal axis 8 extends from the proximal end section end 6 to the distal end section end 7. In the embodiment, this geometric end section longitudinal axis 8 extends through the entire endoscope shaft 2.

A left objective 9, a right objective 10, a left deflection prism 11, a right deflection prism 12, a left image sensor 13, and a right image sensor 14 are arranged in the distal end section 5. The left objective 9, the left deflection prism 11, and the left image sensor 13 form a left optical channel. The right objective 10, the right deflection prism 12, and the right image sensor 14 form a right optical channel. The left objective 9 and the right objective 10 are shown in FIG. 6. The left deflection prism 11, the right deflection prism 12, the left image sensor 13, and the right image sensor 14 form a deflection prism image sensor unit. This is shown in FIGS. 2, 3, 5, 7, and 8.

Light that is coupled into the left optical channel via the left objective 9 enters the left deflection prism 11 via a left entrance surface 15, is reflected a first time at a left first deflection surface 16, reflected a second time at a left second deflection surface 17, and finally exits the left deflection prism 11 at the left exit surface 18. The left image sensor 13 with its light-sensitive sensor surface 19 is attached to the left exit surface 18 so that the light emerging from the left deflection prism 11 falls directly onto the light-sensitive sensor surface 19 of the left image sensor 13.

Light that is coupled into the right optical channel via the right objective 10 enters the right deflection prism 12 via a right entrance surface 20, is reflected a first time at a right first deflection surface 21, reflected a second time at a right second deflection surface 22, and finally exits the right deflection prism 12 at the right exit surface 23. The right image sensor 14 is attached to the right exit surface 23 with its light-sensitive sensor surface 24, so that the light emerging from the right deflection prism 12 falls directly onto the light-sensitive sensor surface 24 of the right image sensor 14.

The left image sensor 13 and the right image sensor 14 are essentially identical. The left deflection prism 11 and the right deflection prism 12 are also essentially identical.

The left entrance surface 15 has the shape of a trapezoid with sides a_{L}, b_{L}, c_{L}, and d_{L} and angles α_{L}=β_{L}=90°, γ_{L}=45°, and δ_{L}=135°. The longer of the two parallel sides a_{L} of the trapezoid forms the boundary to the left exit surface 18. The trapezoid side b_{L} forms the boundary to the left first deflection surface 16. The trapezoid side c_{L} forms the boundary to the left second deflection surface 17. The trapezoid side d_{L} is parallel to a_{L}. a_{L} and b_{L} form an angle α_{L}=90°. b_{L} and d_{L} form an angle β_{L}=90°. a_{L} and c_{L} enclose the angle γ_{L}=45°. d_{L} and c_{L} enclose the angle δ_{L}=135°.

The left first deflection surface 16 and the left entrance surface 15 form an angle ϵ_{L}=45°. Furthermore, the left entrance surface 15 of the left deflection prism and the left second deflection surface 17 form an angle η_{L}=90°. In addition, the left second deflection surface 17 forms an angle θ_{L}=45° with the left exit surface 18. This angle is not shown in the drawing. The left entrance surface 15 is perpendicular to the left exit surface 18.

The same applies to the right deflection prism:

The right entrance surface 20 has the shape of a trapezoid with sides a_{R}, b_{R}, c_{R}, and d_{R} and angles α_{R}=β_{R}=90°, γ_{R}=45°, and δ_{R}=135°. The longer of the two parallel sides a_{R} of the trapezoid forms the boundary to the right exit surface 23. The trapezoid side b_{R} forms the boundary to the right first deflection surface 21. The trapezoid side c_{R} forms the boundary to the right second deflection surface 22. The trapezoid side d_{R} is parallel to a_{R}. a_{R} and b_{R} form an angle α_{R}=90°. b_{R} and d_{R} form an angle β_{R}=90°. a_{R} and c_{R} enclose the angle γ_{R}=45°. d_{R} and c_{R} enclose the angle δ_{R}=135°.

The right first deflection surface 21 and the right entrance surface 20 enclose an angle ϵ_{R}=45°. Furthermore, the right entrance surface 20 of the right deflection prism 12 and the right second deflection surface 22 enclose an angle η_{R}=90°. In addition, the right second deflection surface 22 forms an angle θ_{R}=45° with the right exit surface 23. The right entrance surface 20 is perpendicular to the right exit surface 23. The angles ϵ_{R}, η_{R} and θ_{R} are not shown in the drawing.

The left deflection prism 11 and the right deflection prism 12 are joined together to form the deflection prism unit in such a way that the left entrance surface 15 and the right entrance surface 20 lie in a common geometric plane and together form a rectangle. Furthermore, the left deflection prism 11 and the right deflection prism 12 are joined together in such a way that the left second deflection surface 17 and the right second deflection surface 22 lie against each other and touch each other. The left second deflection surface 17 and the right second deflection surface 22 are both rectangular and have the same size. They lie congruently against each other. There is therefore no protrusion at the deflection surfaces 17 and 22. Since total reflection occurs at the left second deflection surface 17 and at the right second deflection surface, no light from the first deflection prism 11 enters the second deflection prism 12 and vice versa.

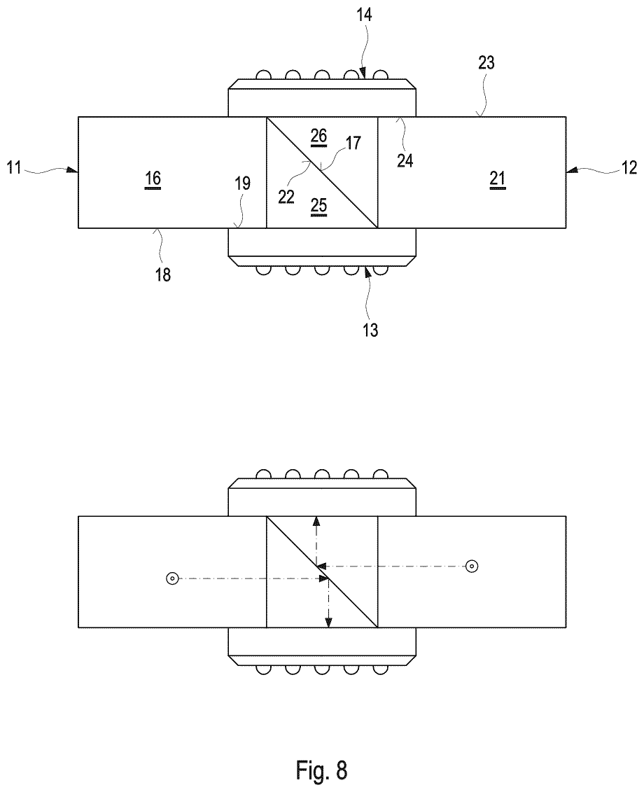

FIG. 8 shows the deflection prism image sensor unit in a top view. In this representation, the upper left cover surface 25 of the left deflection prism 11 and the upper right cover surface 26 of the right deflection prism 12 are visible. The upper left cover surface 25 is parallel to the left entrance surface 15. Furthermore, the upper right cover surface 26 is parallel to the right entrance surface 20.

In the distal end section 5, the deflection prism image sensor unit is inserted in such a way that the left entrance surface 15 and the right entrance surface 20 are aligned perpendicular to the end section longitudinal axis 8.

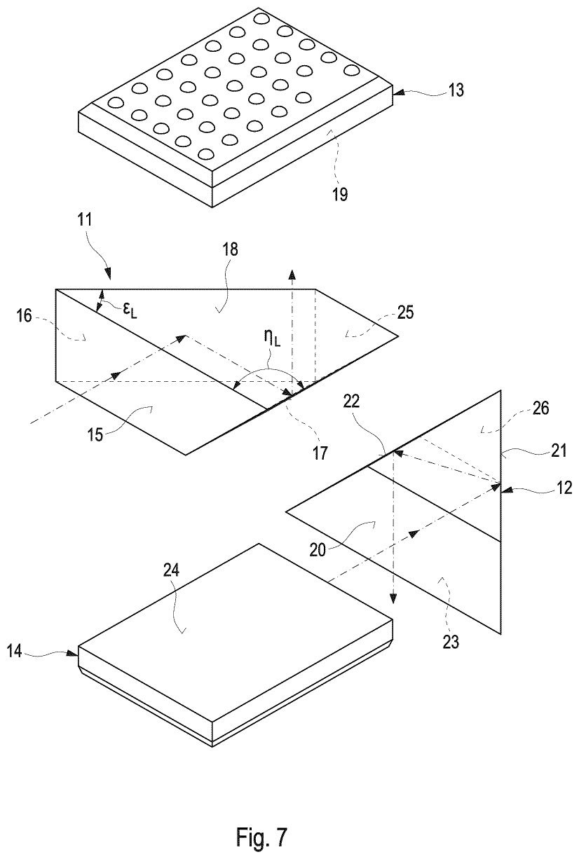

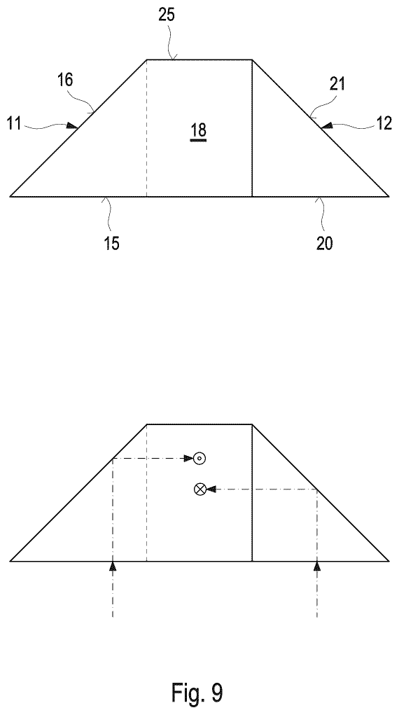

FIGS. 7, 8, and 9 show the beam path of a left light beam that enters the left deflection prism 11 via the left objective 9 parallel to the geometric end section longitudinal axis 8 according to FIG. 1, is deflected twice, and is directed onto the left image sensor 13. FIGS. 7, 8, and 9 also show the beam path of a right light beam that enters the right deflection prism 11 via the right objective 10 parallel to the geometric end section longitudinal axis 8 according to FIG. 1, is deflected twice, and is directed onto the right image sensor 14. The beam path is shown in FIGS. 7, 8, and 9 by dashed lines with arrows. The left light beam, which is incident parallel to the geometric end section longitudinal axis 8, enters the left deflection prism 11 at the left entrance surface 15, which is perpendicular to the geometric end section longitudinal axis. Therefore, the left light beam does not undergo any deflection at the left entrance surface 15. The left light beam is deflected a first time by 90° at the left first deflection surface 16 and a second time by 90° at the left second deflection surface 17. After the second deflection, the light beam exits the left deflection prism 11 at the left exit surface 18 and reaches the left image sensor 13. In FIG. 9, the section of the light beam after the second deflection at the left second deflection surface 17 is shown as a circle with a dot in the center. This representation is intended to illustrate that this section of the light beam runs perpendicular to the drawing plane and in the direction of the viewer. Between the left first deflection surface 16 and the left second deflection surface 17, the left light beam runs perpendicular to the geometric end section longitudinal axis 8. Between the left second deflection surface 17 and the left image sensor 13, the left light beam runs perpendicular to the geometric end section longitudinal axis 8 and perpendicular to the left light beam section between the left first deflection surface 16 and the left second deflection surface 17. The same applies to the right light beam incident parallel to the geometric end section longitudinal axis 8 in relation to the right deflection prism 12 and the right image sensor 14: the right light beam incident parallel to the end section longitudinal axis 8 enters the right deflection prism 12 at the right entrance surface 20 without deflection. It is deflected a first time by 90° at the right first deflection surface 21 and a second time by 90° at the right second deflection surface 22. After the second deflection, the light beam exits at the right exit surface 23 and reaches the right image sensor 14. The right light beam is deflected once by 90° at the right first deflection surface 21 and a second time by 90° at the left second deflection surface 22. After the second deflection, the light beam exits the right deflection prism 12 at the right exit surface 23 and reaches the right image sensor 14. In FIG. 9, the section of the light beam after the second deflection at the right second deflection surface 22 is shown as a circle with a cross. This representation is intended to illustrate that this section of the light beam runs perpendicular to the drawing plane and in the direction opposite to the viewer. Between the right first deflection surface 21 and the right second deflection surface 22, the right light beam runs perpendicular to the geometric end section longitudinal axis 8. Between the right second deflection surface 22 and the right image sensor 14, the right light beam runs perpendicular to the geometric end section longitudinal axis 8 and perpendicular to the right light beam section between the right first deflection surface 21 and the right second deflection surface 22.

All features of the invention can be essential to the invention, both individually and in any combination with each other.

### REFERENCE NUMBERS

1 3D video endoscope
2 Endoscope shaft
3 Endoscope base body
4 Proximal shaft end
5 Distal end section
6 Proximal end section end
7 Distal end section end
8 Geometric end section longitudinal axis
9 Left objective
10 Right objective
11 Left deflection prism
12 Right deflection prism
13 Left image sensor
14 Right image sensor
15 Left entrance surface
16 Left first deflection surface
17 Left second deflection surface
18 Left exit surface
19 Light-sensitive sensor surface
20 Right entrance surface
21 Right first deflection surface
22 Right second deflection surface
23 Right exit surface
24 Light-sensitive sensor surface
25 Left upper cover surface
26 Right upper cover surface

1. A 3D video endoscope
with an endoscope shaft (2) designed as a flexible or rigid, elongated hollow body, with a distal end section (5) of the endoscope shaft (2) having a proximal end section end (6), a distal end section end (7), and a geometric end section longitudinal axis (8) extending from the distal end section end (7) to the proximal end section end (6), with a left optical channel comprising a left objective (9) at the distal end portion end (7), a left optical image transmission system arranged in the distal end section (5), and a left image sensor (13) arranged in the distal end section (5), wherein the left optical image transmission system is designed to transmit an image received by the left objective (9) to the left image sensor (13),
with a right optical channel comprising a right objective (10) at the distal end portion end (7), a right optical image transmission system arranged in the distal end section (5), and a right image sensor (14) arranged in the distal end section (5), wherein the right optical image transmission system is designed to transmit an image received by the right lens (10) to the right image sensor (14),
wherein the left optical image transmission system comprises a left deflection prism (11) which deflects a light beam incident parallel to the geometric end section longitudinal axis (8) by 90° at a left first deflection surface (16) and deflects it by 90° at a left second deflection surface (17) such that the light beam after deflection at the left second deflection surface (17) runs perpendicular to the geometric end section longitudinal axis (8),
wherein the right optical image transmission system comprises a right deflection prism (12) which deflects a light beam incident parallel to the geometric end section longitudinal axis (8) by 90° at a right first deflection surface (21) and deflects it by 90° at a right second deflection surface (22) such that the light beam after deflection at the right second deflection surface (22) runs perpendicular to the geometric end section longitudinal axis (8),
wherein the left deflection prism (11) and the right deflection prism (12) are joined at the left second deflection surface (17) and at the right second deflection surface (22) to form a deflection prism unit,
wherein the left image sensor (13) and the right image sensor (14) are arranged parallel to the geometric end section longitudinal axis (8), and
wherein the deflection prism unit is arranged between the left image sensor (13) and the right image sensor (14).

2. The 3D video endoscope according to claim 1, wherein the left image sensor (13) with its light-sensitive sensor surface (19) is arranged on the surface of the deflection prism unit (11, 12) in such a way that its light-sensitive sensor surface (19) touches the surface of the deflection prism unit (11, 12), and wherein the right image sensor (14) is arranged with its light-sensitive sensor surface (24) in such a way that its light-sensitive sensor surface (24) touches the surface of the deflection prism unit (11, 12).

3. The 3D video endoscope according to claim 1, wherein the deflection prism unit (11, 12), the left image sensor (13) and the right image sensor (14) are combined to form a deflection prism image sensor unit.

4. The 3D video endoscope according to claim 1, wherein a left entrance surface (15) of the left deflection prism (11) faces the left objective (9) and wherein a left exit surface (18) of the left deflection prism (11) faces the left image sensor (13).

5. The 3D video endoscope according to claim 4, wherein the left entrance surface (15) of the left deflection prism (11) has the shape of a trapezium which comprises two right angles α_{L}=β_{L}=90°, an angle with γ_{L}=45°, and an angle with δ_{L}=135°, and wherein the side of the trapezoid that forms a leg of the angles γ_{L} and δ_{L} delimits the left second deflection surface.

6. The 3D video endoscope according to claim 1, wherein a right entrance surface (20) of the right deflection prism (12) faces the right objective (10) and wherein a right exit surface (23) of the right deflection prism (12) faces the right image sensor (14).

7. The 3D video endoscope according to claim 6, wherein the right entrance surface (20) of the right deflection prism (12) has the shape of a trapezium which comprises two right angles α_{R}=β_{R}=90°, an angle with γ_{R}=45°, and an angle with δ_{R}=135°, and wherein the side of the trapezoid which forms one leg of the angles γ_{R} and δ_{R} delimits the right second deflection surface.

8. The 3D video endoscope according to claim 6, wherein, in the deflection prism unit, the left entrance surface (15) of the left deflection prism (11) and the right entrance surface (20) of the right deflection prism (12) lie in a common geometric entrance surface plane, and wherein the two entrance surfaces (15, 20) together form a rectangle.

9. The 3D video endoscope according to claim 4, wherein the left entrance surface (15) of the left deflection prism (11) and the left first deflection surface (16) enclose an angle ϵ_{L}=45°.

10. The 3D video endoscope according to claim 4, wherein the left entrance surface (15) of the left deflection prism (11) and the left second deflection surface (17) form an angle η_{L}=90°.

11. The 3D video endoscope according to claim 4, wherein the left entrance surface (15) of the left deflection prism (11) and the left exit surface (18) of the left deflection prism (11) are perpendicular to each other.

12. The 3D video endoscope according to claim 6, wherein the right entrance surface (20) of the right deflection prism (12) and the right first deflection surface (21) form an angle ϵ_{R}=45°.

13. The 3D video endoscope according to claim 6, wherein the right entrance surface (20) of the right deflection prism (12) and the right second deflection surface (22) enclose an angle η_{R}=90°.

14. The 3D video endoscope according to claim 6, wherein the right entrance surface (20) of the right deflection prism (12) and the right exit surface (23) of the right deflection prism (12) are perpendicular to each other.

15. The 3D video endoscope according to claim 6, wherein, in the deflection prism unit, the left entrance surface (18) of the left deflection prism (11) and the right exit surface (23) of the right deflection prism (12) are parallel to each other.

16. The 3D video endoscope according to claim 1, wherein, in the deflection prism unit, the left first deflection surface (16) and the right first deflection surface (21) are perpendicular to each other.

17. The 3D video endoscope according to claim 1, wherein the left entrance surface (15) of the left deflection prism (11) is perpendicular to the geometric end section longitudinal axis (8), and wherein the right entrance surface (20) of the right deflection prism (12) is perpendicular to the geometric end section longitudinal axis (8).

18. The 3D video endoscope according to claim 1, wherein the deflection prism unit has the outer shape of a mathematical cylinder.

19. The 3D video endoscope according to claim 1, wherein the left deflection prism (11) exhibits total reflection on the left first deflection surface (16) and on the left second deflection surface (17).

20. The 3D video endoscope according to claim 1, wherein the right deflection prism (12) exhibits total reflection at the right first deflection surface (21) and at the right second deflection surface (22).

21. The 3D video endoscope according to claim 1, wherein the left second deflection surface (17) is the same size as the right second deflection surface (22) and wherein the left deflection prism (11) and the right deflection prism (12) are joined together in such a way that the left second deflection surface (17) lie completely against the right second deflection surface (22).

22. The 3D video endoscope according to claim 1, further comprising an image processing device which generates three-dimensional images from left images produced by the left image sensor (13) and from right images produced by the right image sensor (14).
